(19) United States
(12) Patent Application Publication
(10) Pub. No.: US 2023/0212585 A1
(43) Pub. Date: Jul. 6, 2023

(54) Cellular Ablation of HLA-Class I MHC

(71) Applicant: AZTherapies, Inc., Boston, MA (US)

(72) Inventor: Philip G. Ashton-Rickardt, Boston, MA (US)

(21) Appl. No.: 17/919,736

(22) PCT Filed: Apr. 23, 2021
(86) PCT No.: PCT/US2021/028808

§ 371 Date: Oct. 18, 2022

Related U.S. Application Data
(60) Provisional application No. 63/014,344, filed on Apr. 23, 2020.

(51) Int. Cl. C12N 15/113; C12N 15/86; C07K 14/725; A61K 35/17

(52) U.S. Cl. CPC C12N 15/1138; C12N 15/86; C07K 14/7051; A61K 35/17; C12N 2310/14; C12N 2310/531; C12N 2740/15043

(57) ABSTRACT

The invention provides compositions and methods for reducing the immunogenicity of cells for transplant including cell-based immunotherapies. Vectors encoding beta 2 microglobulin (B2M) modifying RNAs along with targeting moieties and other signaling and/or suicide genes allow for efficient production of engineered CAR T-regulatory or other therapeutic cells from any source.

### CROSS-REFERENCE TO RELATED APPLICATIONS

This application claims the benefit of and priority to U.S. Provisional Patent Application No. 63/014,344, filed on Apr. 23, 2020, which is incorporated by reference in its entirety.

### SEQUENCE LISTING

The instant application contains a Sequence Listing with 11 sequences, which has been submitted via EFS-Web and is hereby incorporated herein by reference in its entirety. Said ASCII copy, created on Apr. 21, 2021, is named 48216WO_CRF_sequencelisting.txt, and is 6,287 bytes in size.

### FIELD OF THE INVENTION

The invention provides compositions and methods for cellular ablation of a human leukocyte antigen encoding class I major histocompatibility complex proteins.

### BACKGROUND

The advent of cell-based treatments including stem cell transplants and various immunotherapies have shown significant promise in the treatment of diseases including cancer. In particular, engineered T cells expressing chimeric antigen receptors (CAR-T cells) have entered the spotlight as a new weapon in targeted immunotherapy. Additionally, as discussed in Pat. Pub. WO 2019/190879, incorporated herein by reference, other immune cells including T regulatory lymphocytes (Tregs) can be engineered to express chimeric antigen receptors and used to regulate immune response and inflammation in autoimmune and inflammatory diseases.

These therapies come with their own hurdles and risks related to the immunogenicity of transplanted cells. While the risk of an immune reaction to an intended therapy is generally problematic, in cases where the treatment goal is immunosuppression, as with the aforementioned engineered Tregs, drawing additional immune response is a particular worry. Existing methods for moderating immunogenicity include the use of autologous cells harvested from the patient to be treated. Such methods, however, cause issues with delayed treatment and prevent large scale production.

### SUMMARY

Compositions and methods of the invention use beta 2 microglobulin (B2M) modifying RNA to reduce HLA class I MHC expression in a variety of cells. With reduced class I MHC expression, cells are less likely to be recognized as foreign and induce an unwanted immune response.

Vectors encoding the B2M modifying RNA can be used to transduce cells such as Treg cells to express B2M modifying RNA. B2M modifying RNA can include small interfering RNA (siRNA), or small hairpin RNA (shRNA) can be engineered to target B2M-encoding mRNA and disrupt translation thereof resulting in B2M knockdown cells with reduced class I MHC expression and, therefore, reduced immunogenicity.

The ability to reduce immunogenicity in any cell opens up new cell sources for any treatment or technique relying on cell transplantation (e.g., CAR Treg therapy) and can allow for larger-scale production and stockpiling by avoiding the need for autologous starting material. In certain techniques, such as engineered CAR Tregs, transduction is already required to express the CAR or other targeting moiety in the Treg cell. Accordingly, in preferred embodiments, a single vector encoding both the B2M-modifying RNA and a targeting moiety such as a CAR may be used to produce CAR Tregs with reduced immunogenicity due to B2M knockdown. Such simplified, single-vector approaches further supports larger-scale production.

In certain embodiments, additional elements may be encoded in the vector including, for example, suicide genes and reporter genes useful in providing a safety switch and a means for monitoring expression during cell production and monitoring/tracking of therapeutic cells and their expression profiles after administration. Reporter expression can be particularly useful when used in targeted therapies such as glial-cell-targeted CAR Tregs in order to ensure the desired CNS concentration of the engineered immunosuppressive cells.

As noted, some embodiments include B2M-modified, targeted T regulatory lymphocytes (Tregs) that can be used to modulate immune responses and inflammation through specific targeting to select immune cells and/or select tissues. Such Tregs can be coupled to a chimeric antigen receptor (CAR), antibodies, or functional components thereof (e.g., single-chain variable fragments (scFv)) that specifically recognize and bind various target cells or tissue, the immunosuppressive Tregs or proteins are drawn to inflamed tissue to reduce inflammation and, accordingly, reduce inflammation-related pain and degeneration. The knockdown of B2M expression in such cells, which may be accomplished via transduction with a single vector encoding both the targeting moiety and a B2M modifying RNA, can help avoid unwanted immunogenicity regardless of the source of the engineered Treg cell. The B2M modifying vectors and methods of the invention are compatible with targeted-Treg therapies for treating neurodegenerative disorders as described in Pat. Pub. WO 2019/190879 and for treating other immune and inflammatory diseases. Accordingly, vectors encoding the glial-cell and other cell/tissue targeting moieties described in those applications are contemplated herein.

Aspects of the invention include a vector encoding a beta-2 microglobulin (B2M) modifying RNA and a chimeric antigen receptor (CAR), an antibody, or functional components thereof. In certain embodiments, the vector is a lentiviral vector.

The B2M-modifying RNA may include small interfering RNA (siRNA). The siRNA may comprise SEQ ID NO: 1, SEQ ID NO: 2, SEQ ID NO: 3, SEQ ID NO: 4, and SEQ ID NO: 5. In certain embodiments, the B2M-modifying RNA may include a small hairpin RNA (shRNA). The shRNA may include a sequence selected from the group consisting of SEQ ID NO: 1, SEQ ID NO: 2, SEQ ID NO: 3, SEQ ID NO: 4, and SEQ ID NO: 5 and one or more hairpin loop sequences selected from the group consisting of SEQ ID NO: 6 and SEQ ID NO: 7.

In various embodiments, the vector may be operable to knock-down B2M expression in transduced cells by at least 75% compared to wild type, at least 80% compared to wild type, at least 85% compared to wild type, at least 90% compared to wild type, or at least 95% compared to wild type.

In certain embodiments, the CAR may specifically bind a glial cell marker, an antigen presenting cell (APC) marker, a T helper 1 cell (Th1) marker, or a T helper 17 cell (Th17) marker. In some embodiments, the CAR may specifically bind to a marker specific to cells selected from the group consisting of pancreatic islet cells, pancreatic beta cells, cardiomyocytes, monocytes, macrophages, myeloid cells, intestinal cells, liver cells, kidney cells, kidney podocytes, kidney tubule cells, epithelial cells, salivary gland cells, lung cells, fibroblasts, connective tissue cells, Langerhans cells, keratinocytes, melanocytes, skin cells, hair follicle cells, oligodendrocytes, astrocytes, microglial cells, and hair bulb cells.

Vectors of the invention may further encode a suicide gene and/or a PET reporter gene. In certain embodiments, the vector may encode a TK suicide/PET reporter gene

In certain aspects, the invention may include an engineered cell transduced with a vector encoding a beta-2 microglobulin (B2M) modifying RNA and a chimeric antigen receptor (CAR). The engineered cell may have a B2M expression of 25% or less, 20% or less, 15% or less, 10% or less, or 5% or less that of an otherwise equivalent wild type cell.

The engineered cell may be a stem cell and a lymphocyte. In certain embodiments, the cell may be a regulatory T cell (Treg). The cell may be derived from a donor for allogeneic transplant in a subject. The engineered Treg cell may be derived from an allogeneic donor without the addition of a CAR molecule.

Aspects of the invention may include methods of treating an autoimmune or inflammatory disease in a subject by administering to said subject a therapeutically effective amount of regulatory T cells (Tregs), each expressing a beta-2 microglobulin (B2M) modifying RNA and a chimeric antigen receptor (CAR) that specifically binds a ligand on a surface of a cell in a manner that suppresses an immune response in a subject, thereby treating the autoimmune or inflammatory disease in the subject. The subject may be a human. The Tregs may not be autologous. In various embodiments, the autoimmune or inflammatory disease may be Alzheimer's disease, amyotrophic lateral sclerosis, Batten disease, chronic inflammatory demyelinating polyneuropathy (CIDP), chronic traumatic encephalopathy (CTE), corticobasal degeneration (CBD), Dravet syndrome, Krabbe disease, Guillain-Barré syndrome, Huntington's disease, hypoxic-ischemic encephalopathy, West syndrome, Lewy body dementia, metachromatic leukodystrophy (MLD), Migraine, Multiple System Atrophy (MSA), neuromyelitis optica (NMO), Parkinson's disease, Pelizaeus-Merzbacher disease (PMD), post-traumatic stress disorder (PTSD), prion disease, progressive supranuclear palsy, Rett syndrome, spinal muscular atrophy (SMA), Tourette's syndrome, traumatic brain injury, tropical spastic paraparesis (TSP), abdominal pain, absence seizure, acute spinal cord injury, addiction, amnesia, anxiety disorders, eating disorders, arthralgia, ataxia-telangiectasia (A-T), attention deficit hyperactivity disorder (ADHD), autism, autoimmune encephalitis, back pain, bipolar disorder, bladder pain, Broca's aphasia, cancer pain, cerebral edema, chemotherapy induced pain, cluster headache syndrome, cognitive disorders, cognitive impairment, complex regional pain syndrome, dementia, dental pain, depression, diabetic neuropathic pain, drug-induced dyskinesia, dystonia, encephalomyelitis, epilepsy, epileptic encephalopathy, essential tremor, fatigue, fibromyalgia, hemiplegia, hyperalgesia, inflammatory pain, insomnia, interstitial cystitis, intracerebral hemorrhage, intracranial hypertension, Kennedy's disease, Lennox-Gastaut syndrome, local anesthetic effect, major depressive disorder, MELAS syndrome, meningoencephalitis, nociceptive pain, Morton's metatarsalgia, motor neuron diseases, movement disorders, multifocal motor neuropathy, multiple sclerosis (MS), muscle spasticity, musculoskeletal pain, myalgia, myofascial pain syndrome, narcolepsy, nerve injury, neuropathic pain, neurotoxicity syndromes, obsessive-compulsive disorder, ocular pain, opium withdrawal syndrome, osteoarthritis pain, panic disorders, paralysis, partial seizure, peripheral nerve injury, pervasive developmental disorder (PDD), polymyalgia rheumatica (PMR), postherpetic neuralgia, post-operative pain, primary progressive multiple sclerosis (PPMS), psychosis, radiculopathy, relapsing multiple sclerosis (RMS), relapsing remitting multiple sclerosis (RRMR), restless legs syndrome, rheumatoid arthritis pain, schizoaffective disorder, schizophrenia, sciatica, secondary progressive multiple sclerosis (SPMS), sleep disorders, smoking cessation, social anxiety disorder, spasmodic torticollis (cervical dystonia), spinal cord disorders, stiff-person syndrome (SPS), tauopathies, tendon and ligament pain, tonic-clonic (grand mal) seizure, trigeminal neuralgia, upper limb muscle spasticity, vascular dementias, vasomotor symptoms (non-menopausal), and visceral pain, type 1 diabetes, transplant, cardiomyositis, inflammatory bowel disease, ulcerative colitis, Crohn's disease, GVHD, Celiac disease, autoimmune hepatitis (AIH), primary sclerosing cholangitis (PSC); primary biliary cirrhosis (PBC), focal segmented glomerular sclerosis (FSGS), systemic lupus erythematosus, cutaneous lupus erythematosus, lupus nephritis, systemic scleroderma, membranous glomerular nephropathy (MGN), membranous nephropathy (MN), minimal change disease (MCD); IgA nephropathy, ANCA-associated vasculitis (AAV), Sjogren's syndrome, scleroderma, systemic sclerosis (SSc), vitiligo, non-alcoholic fatty acid liver disease (NAFLD), non-alcoholic steatohepatitis (NASH), alopecia areata, COVID-19 or other inflammatory diseases resulting from virus infection.

### BRIEF DESCRIPTION OF THE DRAWINGS

FIG. 1 illustrates a B2M-modified, targeted CAR-Treg.

FIG. 2 illustrates median fluorescence FACS results with an anti-B2M stain for HEK-293 cells transduced with various shRNA-encoding vectors.

FIG. 3 illustrates median fluorescence FACS results with an anti-HLA ABC stain for HEK-293 cells transduced with various shRNA-encoding vectors.

FIGS. 4A-4B show mean fluorescent intensity for HEK-293 cells transduced with various shRNA-encoding, GFP+ vectors and stained with anti-B2M or anti-HLA ABC.

FIGS. 5A-5D show GFP-measured transduction efficiency for HEK-293 cells transduced with various shRNA-encoding vectors and the intensity of staining for anti-B2M and anti-HLA ABC on GFP+ and GFP-cells.

FIGS. 6A-6D show GFP-measured transduction efficiency for HEK-293 cells transduced with various shRNA-encoding vectors and the intensity of staining with an IgG1 PE/IgG1 APC isotype control antibody.

FIG. 7 shows a sequence map of shRNA target locations across the B2M gene.

FIG. 8 illustrates the production of universal donor (UD) Tregs with or without a CAR or other targeting component. The starting cell types are natural (n) Tregs (CD4⁺CD25⁺CD127⁻) purified from peripheral blood mononuclear leukocytes (PBMCs) from normal donor blood. nTregs can be transduced with lentivirus vectors encoding a B2M-modifying RNA and, optionally, a CAR B2M alone. Both types of engineered nTregs will retain the expression of endogenous T cell receptors (TCR) and will have down-regulated B2M expression and ablation of HLA-class I MHC. Only nTregs transduced with CAR/shRNA B2M sequences will express CAR specific for protein targets on defined cell types. The ablation of HLA-class I MHC will protect Tregs from immune attack from allo-reactive immune cells after adoptive transfer to MHC-miss matched patients. Immunosuppression by UD CAR-Tregs will be triggered by either the recognition of allo-HLA class II MHC through endogenous TCR and the recognition of defined cell surface target protein by the CAR. Immunosuppression by UD Tregs will only be triggered by the recognition of allo-class II MHC by endogenous TCR.

### DETAILED DESCRIPTION

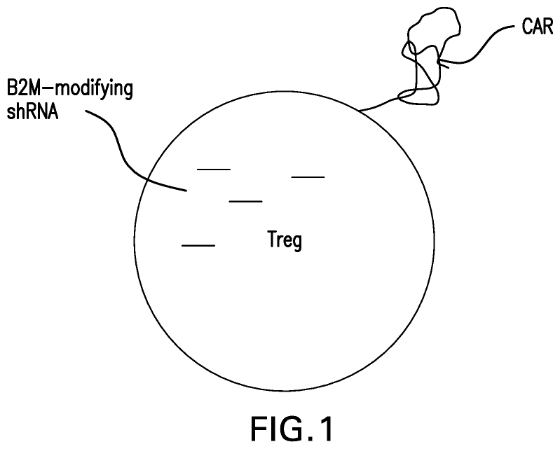

Compositions and methods of the invention relate to the reduction of immunogenicity in various cells through transduction with a vector encoding B2M-modifying RNA. In preferred embodiments, the B2M-modifying RNA is an siRNA or shRNA that disrupts the expression of B2M through RNA interference to thereby reduce or prevent the display of MCH class I proteins on the cell surface. Accordingly, engineered cells of the invention can be derived from more readily available non-autologous sources without increased risk of inducing an immune response in the recipient patient. When combined with cell-based immunotherapies such as CAR Treg immunosuppression, a single vector may be used to induce expression of the B2M-modifying RNA as well as the targeting-moiety (e.g., CAR) and other genes such as reporting or suicide genes. FIG. 1 illustrates an engineered Treg cell of the invention expressing a cell-specific CAR and B2M-modifying shRNA to reduce MHC class I cell surface display.

“Major histocompatibility complex antigens” (“MHC”, also called “human leukocyte antigens”, HLA) are protein molecules found on the surface of cells and are critical in cell-based immune responses through the display of foreign proteins to cytotoxic T cells to induce immune attack. HLA antigens are divided into two main classes: MHC class I and MHC class II. HLAs corresponding to MHC class I (HLA-A, HLA-B, and HLA-C) allow cells to be recognized as self while HLAs corresponding to MHC class II (DP, DM, DO, DQ and DR) are involved in reactions between lymphocytes and antigen presenting cells. Both have been implicated in the rejection of transplanted organs. See U.S. Pat. No. 9,997,807, incorporated herein by reference.

B2M is a component of MHC class I proteins along with α1, α2, and α3 proteins and is essential for MHC class I peptide presentation. B2M lies beside the α3 chain on the cell surface and has no transmembrane region. Studies have shown that B2M is necessary for cell surface expression of MHC class I and stability of the peptide binding groove such that the absence of B2M results in very limited amounts of MHC class I detectable on cell surfaces. Accordingly, reduction or elimination of B2M expression in a cell can help avoid recognition of the cell as foreign and reduce immunogenicity. Class I MHC ablation through interference of B2M expression can therefore open up new sources of non-autologous cell sources for use in engineering cells for transplant.

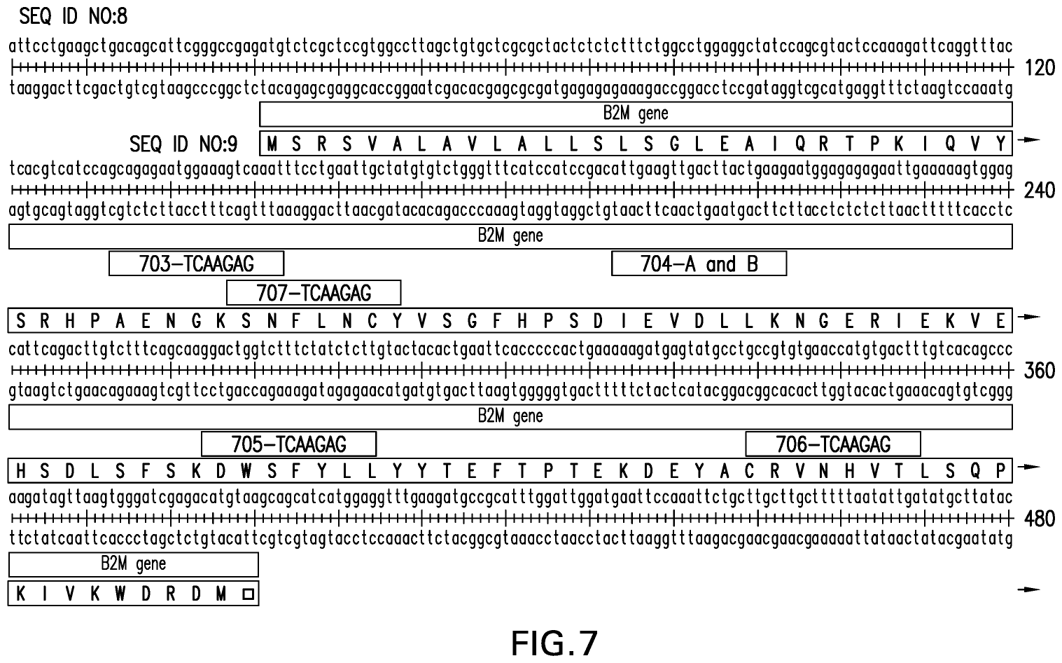

The nucleotide sequence for the human B2M gene (SEQ ID NO: 8) as well as the peptide sequence of the B2M protein (SEQ ID NO: 9; UniProtKB—P61769 (B2MG_HUMAN)) are shown in FIG. 7. Human B2M including adjacent sequences is shown in SEQ ID NO: 10 below (NCBI Reference Sequence: NM_004048):

| | |
|---|---|
| 1 | attcctgaag ctgacagcat tcgggccgag atgtctcgct ccgtggcctt agctgtgctc |
| 61 | gcgctactct ctctttctgg cctggaggct atccagcgta ctccaaagat tcaggtttac |
| 121 | tcacgtcatc cagcagagaa tggaaagtca aatttcctga attgctatgt gtctgggttt |
| 181 | catccatccg acattgaagt tgacttactg aagaatggag agagaattga aaaagtggag |
| 241 | cattcagact tgtctttcag caaggactgg tctttctatc tcttgtacta cactgaattc |
| 301 | acccccactg aaaaagatga gtatgcctgc cgtgtgaacc atgtgacttt gtcacagccc |
| 361 | aagatagtta agtgggatcg agacatgtaa gcagcatcat ggaggtttga agatgccgca |
| 421 | tttggattgg atgaattcca aattctgctt gcttgctttt taatattgat atgcttatac |
| 481 | acttacactt tatgcacaaa atgtagggtt ataataatgt taacatggac atgatcttct |
| 541 | ttataattct actttgagtg ctgtctccat gtttgatgta tctgagcagg ttgctccaca |
| 601 | ggtagctcta ggagggctgg caacttagag gtggggagca gagaattctc ttatccaaca |
| 661 | tcaacatctt ggtcagattt gaactcttca atctcttgca ctcaaagctt gttaagatag |
| 721 | ttaagcgtgc ataagttaac ttccaattta catactctgc ttagaatttg ggggaaaatt |
| 781 | tagaaatata attgacagga ttattggaaa tttgttataa tgaatgaaac attttgtcat |
| 841 | ataagattca tatttacttc ttatacattt gataaagtaa ggcatggttg tggttaatct |
| 901 | ggtttatttt tgttccacaa gttaaataaa tcataaaact tgatgtgtta tctcttatat |
| 961 | ctcactccca ctattacccc tttattttca aacagggaaa cagtcttcaa gttccacttg |
| 1021 | gtaaaaaatg tgaacccctt gtatatagag tttggctcac agtgtaaagg gcctcagtga |
| 1081 | ttcacatttt ccagattagg aatctgatgc tcaaagaagt taaatggcat agttggggtg |
| 1141 | acacagctgt ctagtgggag gccagccttc tatattttag ccagcgttct ttcctgcggg |
| 1201 | ccaggtcatg aggagtatgc agactctaag agggagcaaa agtatctgaa ggatttaata |
| 1261 | ttttagcaag gaatagatat acaatcatcc cttggtctcc ctgggggatt ggtttcagga |
| 1321 | ccccttcttg gacaccaaat ctatggatat ttaagtccct tctataaaat ggtatagtat |
| 1381 | ttgcatataa cctatccaca tcctcctgta tactttaaat catttctaga ttacttgtaa |
| 1441 | tacctaatac aatgtaaatg ctatgcaaat agttgttatt gtttaaggaa taatgacaag |
| 1501 | aaaaaaaagt ctgtacatgc tcagtaaaga cacaaccatc cctttttttc cccagtgttt |
| 1561 | ttgatccatg gtttgctgaa tccacagatg tggagcccct ggatacggaa ggcccgctgt |
| 1621 | actttgaatg acaaataaca gatttaaaat tttcaaggca tagttttata cctga |

The B2M protein (SEQ ID NO: 9) includes a signaling portion

(SEQ ID NO: 11)
MSRSVALAVLALLSLSGLEA.

In preferred embodiments, siRNA or shRNA is used for RNA interference with B2M expression in transduced cells. Select siRNA and shRNA targets within the B2M gene were selected as mapped in FIG. 7. Examples of genetic ablation of B2M to reduce immunogenicity have been shown in previous work. See, Chang, et al., 2014, Broad T-Cell Receptor Repertoire in T-Lymphocytes Derived from Human Induced Pluripotent Stem Cells, PLoS ONE 9(5); Aldrich, et al., 1994, Positive selection of self- and alloreactive CD8+ T cells in TAP1 mutant mice, PNAS, 91:6525-6528; Wang, et al., 2015, Targeted Disruption of the B2-Microglobulin Gene Minimizes the Immunogenicity of Human Embryonic Stem Cells, Stem Cells Translational Medicine, 4:1234-1245; Zijlstra, et al., 1990, β2-Microglobulin Deficient Mice Lack CD4⁻8⁺ Cytolytic T Cells, Nature, 344:742-746; U.S. Pat. Pub. No. 2019/0233797; the content of each of which is incorporated herein by reference.

The present invention provides new B2M-modifying RNA sequences and vectors encoding B2M-modifying RNA along with additional CAR, reporting, and suicide genes for efficient production of targeted cells for cell-based immunotherapy and other treatments, from any source, with reduced immunogenicity. B2M-modifying vectors of the invention may knock-down B2M expression in transduced cells by at least 75% compared to wild type, at least 80% compared to wild type, at least 85% compared to wild type, at least 90% compared to wild type, or at least 95% compared to wild type.

In various embodiments, the B2M-modifying RNA may be siRNA, shRNA, microRNA, or single stranded interfering RNA. In preferred embodiments, the B2M-modifying RNA is siRNA or shRNA. SiRNA is double-stranded non-coding RNA, generally 20-25 base pairs in length. It interferes with the expression of specific genes with complementary nucleotide sequences by degrading mRNA after transcription and thereby preventing translation. In certain embodiments, the vector sequences encoding the B2M-modifying siRNA comprise one or more of the following sequences:

SEQ ID NO: 1-703-
GCAGAGAATGGAAAGTCAAAT
SEQ ID NO: 2-704-
ATTGAAGTTGACTTACTGAAG
SEQ ID NO: 3-705-
GGACTGGTCTTTCTATCTCTT
SEQ ID NO: 4-706-
GCCGTGTGAACCATGTGACTT
SEQ ID NO: 5-707-
GTCAAATTTCCTGAATTGCTA

B2M-modifying vectors encoding the above siRNA sequences or shRNA sequences comprising those sequences may be referred to herein by the corresponding ID number (e.g., 703, 704, 705, 706, and 707). Vector 728 (discussed in the Examples) comprises a scrambled siRNA sequence included as a control and is not complementary to any target sequence in the B2M mRNA. The human B2M gene sequence is shown in FIG. 7 along with the target regions for each of the above-referenced siRNA sequences.

ShRNA is an artificial RNA molecule with a tight hairpin turn that can be used to silence target gene expression via RNA interference. ShRNAs can be incorporated into plasmid vectors and integrated into genomic DNA for stable expression, producing a longer knockdown effect of the target mRNA. In application, the shRNA molecules are processed within the cell to form siRNA which in turn knock down gene expression. In certain embodiments, hairpin loops such as one of the following may be appended to the siRNA sequences to produce a B2M-modifying shRNA of the invention:

SEQ ID NO: 6-
TCAAGAG
SEQ ID NO: 7-
CTCGAG

With respect to the examples discussed below, vectors 703, 704a, 705, 706, and 707 comprise their respective siRNA sequence along with the hairpin sequence in SEQ ID NO: 6. Vector 704b, discussed in the examples, comprises the siRNA sequence in SEQ ID NO: 2 along with the hairpin loop in SEQ ID NO: 7. The present invention recognizes that certain variations of the sequences provided herein may still exhibit desired levels of B2M-expression interference. Accordingly, in various embodiments, siRNA or shRNA may comprise a sequence having at least 50%, at least 60%, at least 70%, at least 80%, at least 90%, or at least 95% identity to SEQ ID NOS: 1-7.

In various embodiments, vectors may include reporter genes. Reporter genes encode identifiable markers such that their detection in a cell confirms expression of the gene. Reporter genes may provide a measurable signal when expressed that can be further used to quantify expression of the gene and to infer transduction efficiency of the vector in which they are included. Commonly used reporter genes include those that provide a visual signal such as green fluorescent protein (GFP) that can be readily observed and measured. In certain embodiments, vectors of the invention may include Positron Emission Tomography (PET) reporter genes. See Yaghoubi, et al., 2012, Positron Emission Tomography Reporter Genes and Reporter Probes: Gene and Cell Therapy Applications, Teranostics, 2(4):374-391, incorporated herein by reference. The use of PET reporter genes allows for non-invasive imaging using a PET scan to determine expression and, therefore, transduction efficiency. Furthermore, when used in targeted cell-based therapies such as CAR-Treg treatments, PET reporter expression can be used to non-invasively monitor localization of the administered cells to verify that the cells are being properly concentrated in the target tissue. For example, when expressed in CNS-glial-cell-targeting CAR-Tregs, PET reporters can be used in conjunction with PET scans of the brain to verify that the CAR-Tregs are crossing the blood-brain barrier and reaching the target glial cells.

In certain embodiments, vectors may further include suicide genes. Suicide genes can cause a cell to kill itself through apoptosis upon activation. Suicide genes can be incorporated as a safety switch to selectively kill gene-modified cells in case of an adverse reaction. They can accordingly be useful in cell-based immunotherapies such as CAR-Treg immunosuppression. In preferred embodiments, a TK suicide/PET reporter gene may be included in the vector to provide a dual function of PET reporting and suicide safety switch. See, Gschweng, et al., 2014, HSV-sr39TK positron emission tomography and suicide gene elimination of human hematopoietic stem cells and their progeny in humanized mice, Cancer Res., 74(18):5173-5183, incorporated herein by reference.

B2M modified Tregs and other cells may be engineered by known methods for preparing CAR-T cells or other engineered cells. Treg cells may be isolated from any source due to the subsequent class I MHC ablation. The genes of the Treg cells can then be modified through known techniques such as electroporation, viral vectors, or other forms of transfection with nucleic acids encoding one or more of a B2M-modifying RNA, an engineered chimeric antigen receptor of choice, a reporter gene, and a suicide gene. Engineered cells can then be experimentally verified before introduction into the patient's system for treatment. In preferred embodiments, a lentiviral vector is used to transduce cells with B2M-modifying RNA and various targeting, signaling, and suicide genes. See, Elegheert, et al., 2018, Lentiviral transduction of mammalian cells for fast, scalable and high-level production of soluble and membrane proteins, Nature Protocols, 13, 2991-3017, incorporated herein by reference.

In preferred embodiments, the transduced cell may be a stem cell or a lymphocyte such as a Treg. Engineered Tregs of the invention may be used to treat a variety of autoimmune and/or inflammatory diseases including various neurodegenerative diseases. Engineered cells can target cell-specific markers to draw Tregs to target immune cells or target tissues to disrupt autoimmune attacks and inflammation that contribute to the symptoms of a large variety of diseases.

As noted above, in addition to B2M-modifying RNA, reporter, and/or suicide genes, vectors may encode chimeric antigen receptors (CAR), antibodies, or single-chain variable fragments (scFv) that specifically bind target cell markers. Target cells may be immune cells such as APCs or T helper cells (Th1 or Th17) or may be specific tissues in which suppression of immune response is desired. Exemplary target cells, ligands, and targeting moieties for treating neurodegenerative, autoimmune, and/or inflammatory diseases are described in Pat. Pub. WO 2019/190879 and the present invention contemplates incorporation of such targeting moieties and treatment methods in B2M-modifying vectors described herein.

In various embodiments, vectors may encode CARs, scFvs, or antibodies to target specific cells or tissues. CARs are engineered receptors that can provide specificity to immune effector cells (T cells). CARs have been used to confer tumor cell specificity to cytotoxic T lymphocytes for use in cancer immunotherapies. See, Couzin-Frankel, 2013, Cancer immunotherapy, Science, 342(6165):1432-33; Smith, et al., 2016, Chimeric antigen receptor (CAR) T cell therapy for malignant cancers: Summary and perspective, Journal of Cellular Immunotherapy, 2(2):59-68; the contents of each of which are incorporated herein by reference. Using similar principles, compounds and methods of the invention include engineering CARs that are specific to markers found on the aforementioned immune cells or other specific cell types but, instead of grafting the cell-specific CARs to cytotoxic T cells, they are grafted onto engineered immunosuppressive Tregs. CAR-Tregs of the invention may express multiple chimeric antigen receptors targeting the same or two or more different cell markers.

ScFvs are fusion proteins including variable regions of the heavy (V_{H}) and light chains (V_{L}) of immunoglobulins. ScFvs may be created by cloning V_{H} and V_{L} genes of mice or other animals immunized with the desired target molecule. The V_{H} and V_{L} genes can then be expressed in multiple orientations and with various linkers to form a variety of scFvs which may then be experimentally verified to provide desired stability, expression levels, and binding affinity for specific markers or cells.

Antibodies targeting cell markers can be produced by methods known in the art including commercially available services for producing custom antibodies from, for example, Pacific Immunology (San Diego, Calif.) or ABclonal (Woburn, Mass.).

For the production of engineered Tregs starting cell type are natural (n) Tregs (CD4⁺CD25⁺CD127⁻) may be used. The nTregs can be purified from peripheral blood mononuclear leukocytes (PBMCs) from normal donor blood. CAR genes can be introduced into nTregs by transduction with lentivirus vectors in cis with shRNA against B2M. In certain embodiments, targeting CAR genes may be omitted such that the nTregs are singly engineered with an shRNA against B2M on lentivirus vectors. With or without CAR expression, the engineered nTregs should retain the expression of endogenous T cell receptors (TCR) and exhibit down-regulation of B2M and ablation of HLA-class I MHC. Only nTregs transduced with CAR/shRNA B2M sequences express CAR specific for protein targets on defined cell types. The ablation of HLA-class I MHC can protect Tregs from immune attack from allo-reactive immune cells after adoptive transfer to MHC-mismatched patients. Immunosuppression by UD CAR-Tregs can be triggered by either the recognition of allo-HLA class II MHC through endogenous TCR, or the recognition or defined cell surface target protein by the CAR. Alternatively, immunosuppression by UD Tregs cannot be triggered by CAR recognition as no CAR is expressed and are instead triggered by the recognition of allo-class II MHC by endogenous TCR.

Regulatory T cells or Tregs modulate the immune system and generally downregulate the induction and proliferation of effector T cells. Tregs prevent auto-immune responses and aid in the discrimination of self and non-self by the immune system. Regulatory T cells produce inhibitory cytokines including Transforming growth factor beta, Interleukin 35, and Interleukin 10 and can induce other cell types to express interleukin-10. Tregs can also produce Granzyme B, which in turn can induce apoptosis of effector cells. Tregs also function through reverse signaling through direct interaction with dendritic cells and the induction of immunosuppressive indoleamine 2,3-dioxygenase. Tregs can also downregulate immune response through the ectoenzymes CD39 and CD73 with the production of immunosuppressive adenosine. Tregs also suppress immune response through direct interactions with dendritic cells by LAG3 and by TIGIT. Another control mechanism is through the IL-2 feedback loop. Another mechanism of immune suppression by Tregs is through the prevention of co-stimulation through CD28 on effector T cells by the action of the molecule CTLA-4.

A B2M-modified CAR-Treg of the invention may be incorporated into carrier systems containing one or more of the therapeutic compounds described herein. In certain embodiments, the carrier system can be a nanoparticle that includes disulfide-crosslinked polyethyleneimine (CLPEI) and a lipid. The lipid may be a bile acid, such as cholic acid, deoxycholic acid, and lithocholic acid. Such carrier systems are described further in the Examples below. Other exemplary carrier systems are described for example in Wittrup et al. (Nature Reviews/Genetics, 16:543-552, 2015), the content of which is incorporated by reference herein in its entirety.

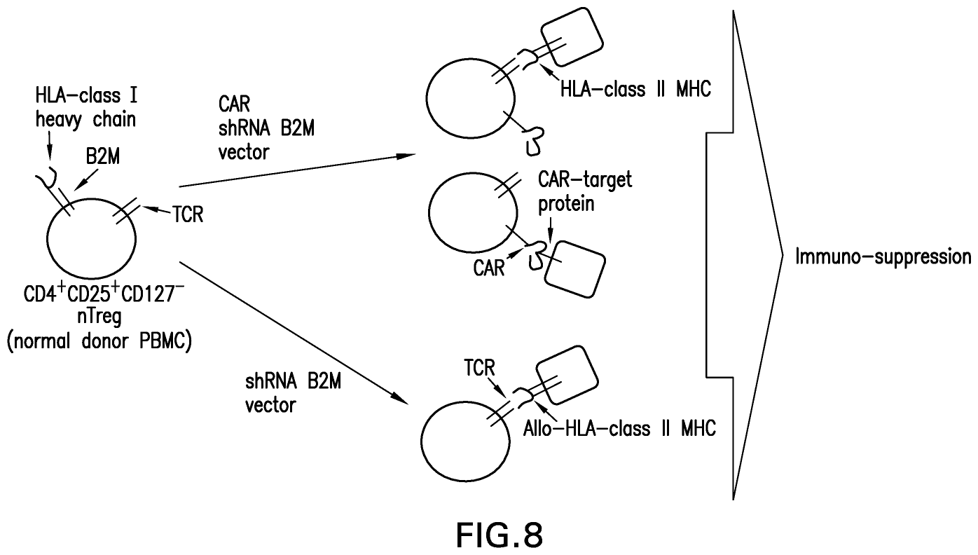

FIG. 8 illustrates the production of universal donor (UD) CAR-Tregs or Tregs. The starting cell type may be natural (n) Tregs (CD4⁺CD25⁺CD127⁻) purified from peripheral blood mononuclear leukocytes (PBMCs) from normal donor blood. nTregs can be transduced with lentivirus vectors encoding either CAR and shRNA against B2M or the shRNA alone. Both types of engineered nTregs should retain expression of endogenous T cell receptors (TCR) and, therefore, endogenous Treg functionality. Both types of engineered nTregs should also exhibit B2M down-regulation and ablation of HLA-class I WIC. Additionally, the nTregs transduced with CAR/shRNA B2M sequences can express CARs specific for protein targets on defined cell types. The ablation of HLA-class I WIC protects the engineered Tregs from immune attack from allo-reactive immune cells after adoptive transfer to WIC-miss matched patients. Immunosuppression by UD CAR-Tregs can be triggered by either the recognition of allo-HLA class II MHC through endogenous TCR and the recognition of defined cell surface target protein by the CAR. Immunosuppression by UD Tregs may only be triggered by the recognition of allo-class II MHC by endogenous TCR.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one a therapeutic compound of the invention and/or derivative thereof, in combination with a pharmaceutically acceptable carrier.

The effective dosage of each agent can readily be determined by the skilled person, having regard to typical factors each as the age, weight, sex and clinical history of the patient. In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the compounds of the present invention, or functional derivatives thereof. An “effective amount” is the amount as defined herein in the definition section and refers to an amount effective, at dosages and for periods of time necessary, to achieve the desired therapeutic result, e.g., a diminishment or prevention of effects associated with neuropathic and/or inflammatory pain. A therapeutically effective amount of a compound of the present invention or functional derivatives thereof may vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to, or at an earlier stage of disease, the prophylactically effective amount may be less than the therapeutically effective amount. A prophylactically or therapeutically effective amount is also one in which any toxic or detrimental effects of the compound are outweighed by the beneficial effects.

Dosage regimens may be adjusted to provide the optimum desired response (e.g. a therapeutic or prophylactic response). For example, a single bolus may be administered, several divided doses may be administered over time or the dose may be proportionally reduced or increased as indicated by the exigency of the therapeutic situation. It is especially advantageous to formulate parenteral compositions in dosage unit form for ease of administration and uniformity of dosage. Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the patient.

The term “dosage unit” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the compound, and (b) the limitations inherent in the art of compounding such an active compound for the treatment of sensitivity in individuals.

In some embodiments, therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount is sufficient to reduce or inhibit neuropathic and/or inflammatory pain in a subject. In some embodiments, the therapeutically effective amount is sufficient to eliminate neuropathic and/or inflammatory pain in a subject.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the compounds of the invention or functional derivatives thereof, and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising one or more compounds of the invention or functional derivatives thereof are optionally tested in one or more appropriate in vitro and/or in vivo animal models of disease, such as models of neuropathic and/or inflammatory pain, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of compounds of the invention or functional derivatives thereof at various concentrations, e.g., as applied to the mass and overall health of the patient. Administration can be accomplished via single or divided doses.

Administering typically involves administering pharmaceutically acceptable dosage forms, which means dosage forms of compounds described herein, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety. Administering may be carried out orally, intradermally, intramuscularly, intraperitoneally, intravenously, subcutaneously, or intranasally. Compounds may be administered alone or with suitable pharmaceutical carriers, and can be in solid or liquid form, such as tablets, capsules, powders, solutions, suspensions, or emulsions.

A pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874 (the content of each of which is incorporated by reference herein in its entirety), to form osmotic therapeutic tablets for control release.

Formulations for oral use may also be presented as hard gelatin capsules in which the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Formulations may also include complexes of the parent (unionized) compounds with derivatives of β-cyclodextrin, especially hydroxypropyl-β-cyclodextrin.

An alternative oral formulation can be achieved using a controlled-release formulation, where the compound is encapsulated in an enteric coating.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Each active agent may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions are suitable. Topical application includes the use of mouth washes and gargles.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of humans and lower animals without undue toxicity, irritation, allergic response, and the like, and are commensurate with a reasonable benefit/risk ratio.

### EXAMPLES

### Example 1—B2M Knockdown in HEK293 Cells

HEK293 cells were transduced with lentiviral vectors encoding shRNA sequences 703-707 and 728 (comprising siRNA SEQ ID NOS: 1-5 with the hairpin loop of SEQ ID NO: 6 or 7) as identified above as well as a GFP reporter gene for monitoring transduction efficiency. The constructs had a 90% or better transduction as measured by GFP.

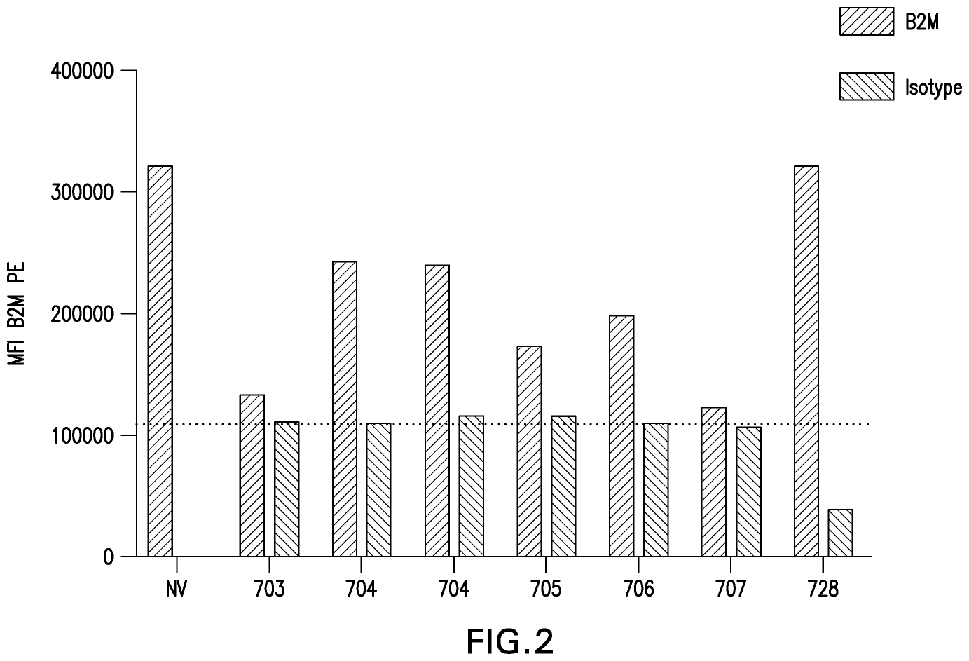

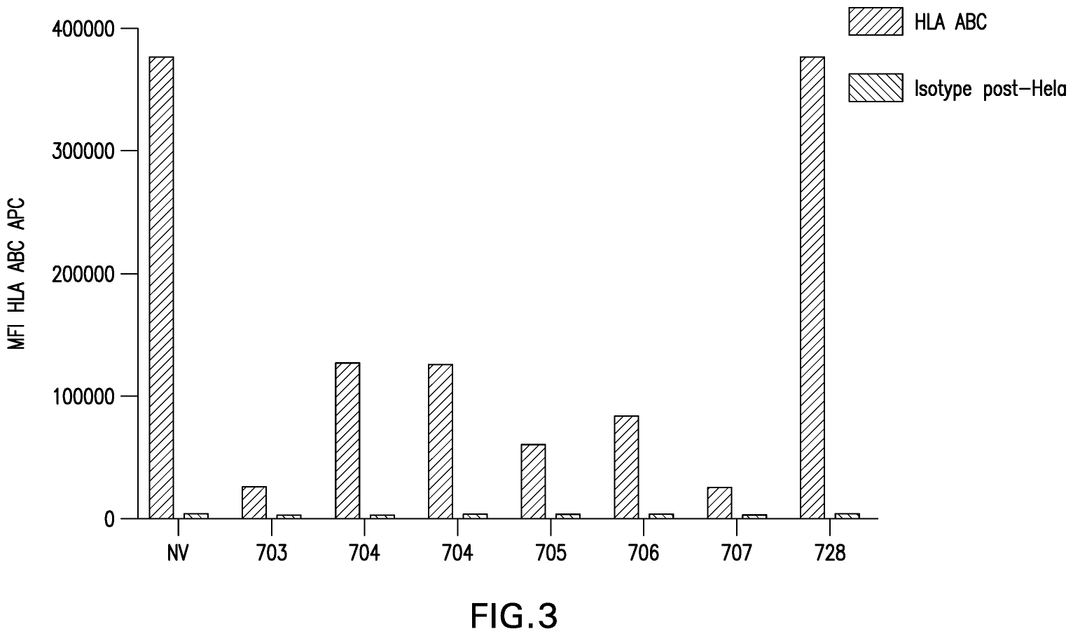

Cells were stained and fluorescence activated cell sorting was used to measure the knockdown effect of the various shRNA vectors compared to controls with no transduction (NV) and a scrambled shRNA-encoding vector (728). FIG. 2 shows median fluorescence FACS results with an anti-B2M stain and FIG. 3 shows median fluorescence FACS results with an anti-HLA ABC stain. As shown, SEQ ID NOS: 1-5 (703-707) each had a significant knockdown effect on both surface B2M and HLA-A/B/C class I MHC expression compared to the two controls. Vectors 703 (encoding SEQ ID NO: 1) and 707 (encoding SEQ ID NO: 5) exhibited the most significant knockdown effect. Furthermore, the B2M knockdown levels were consistent with the HLA knockdown levels across the various vectors.

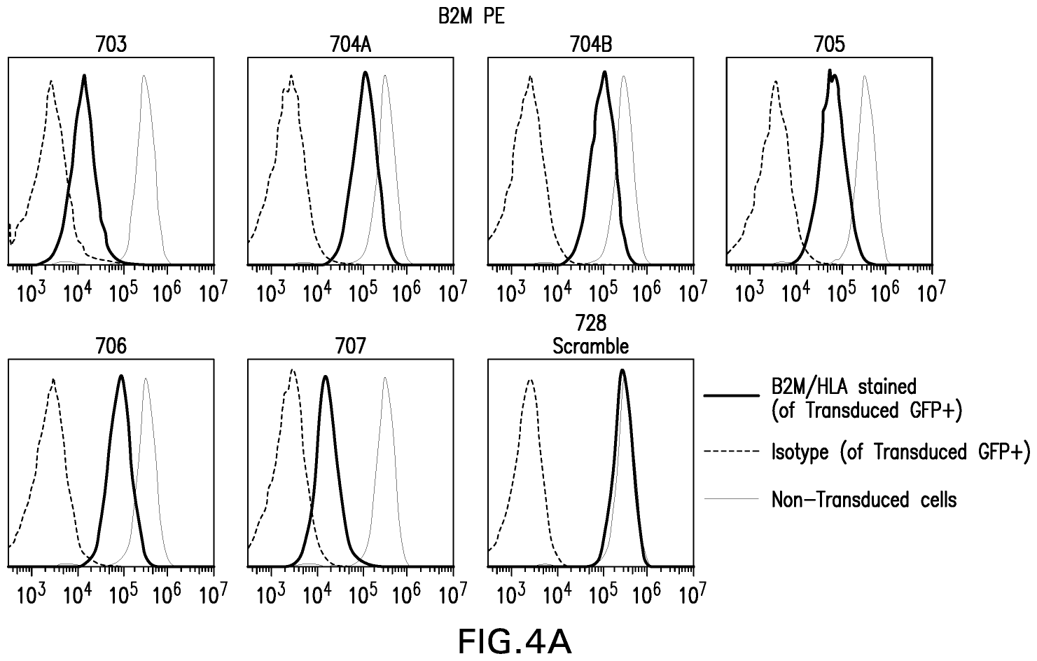

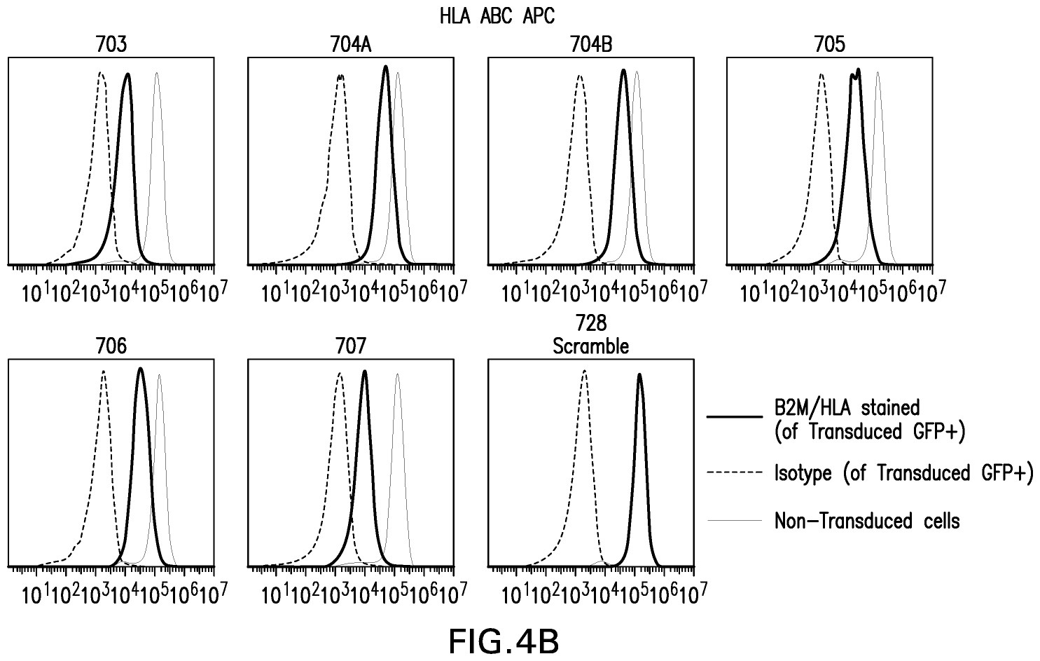

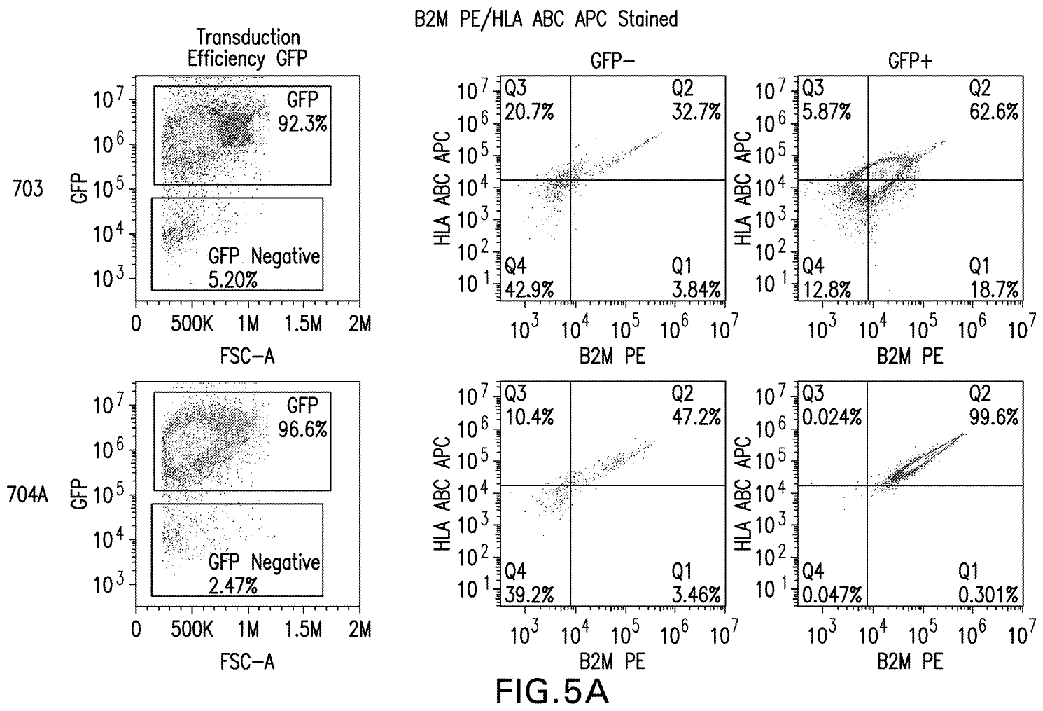

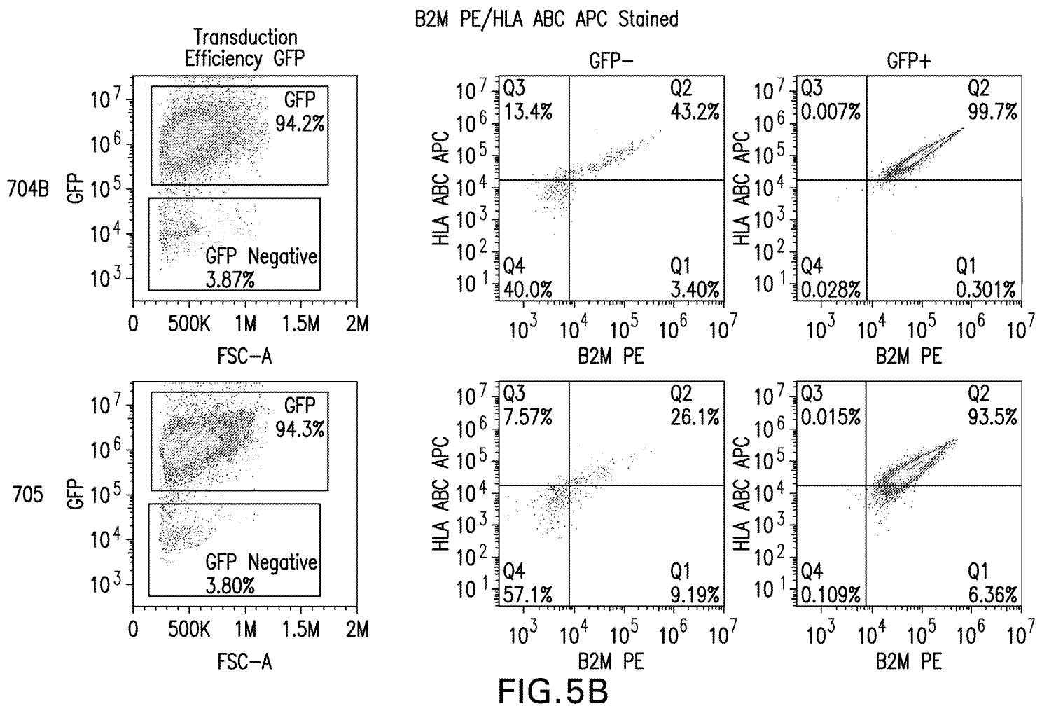

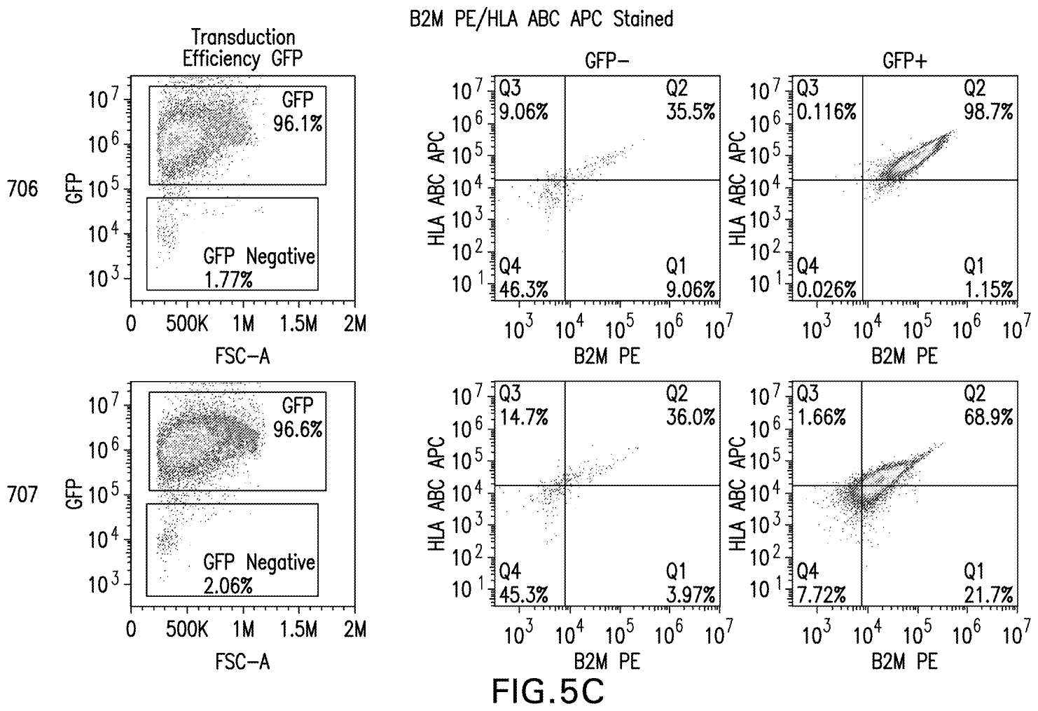

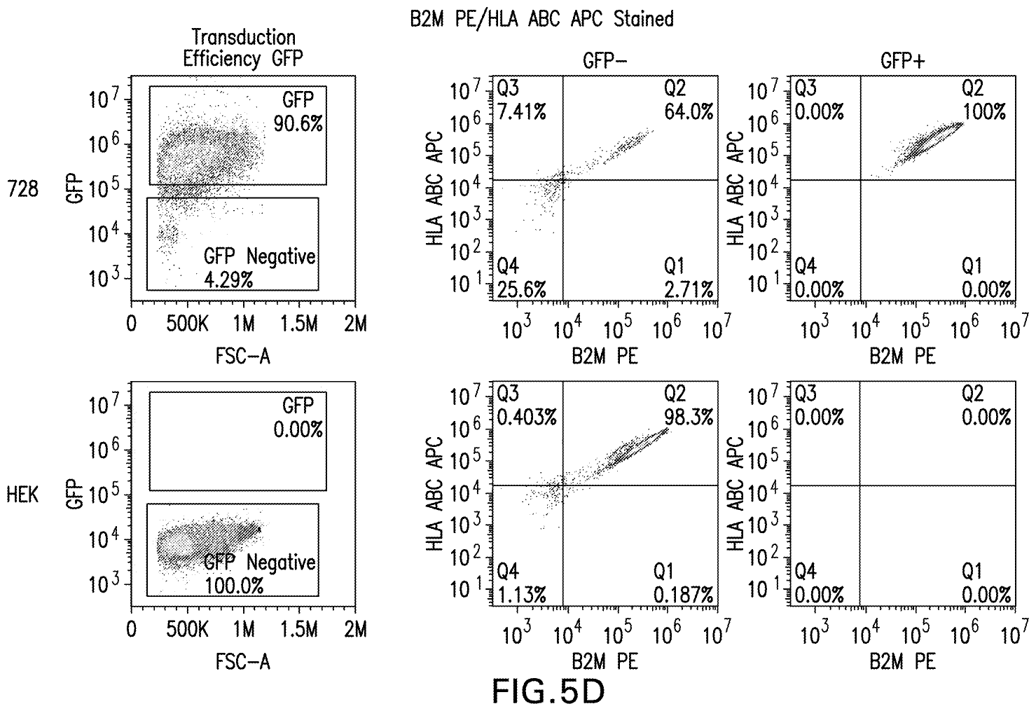

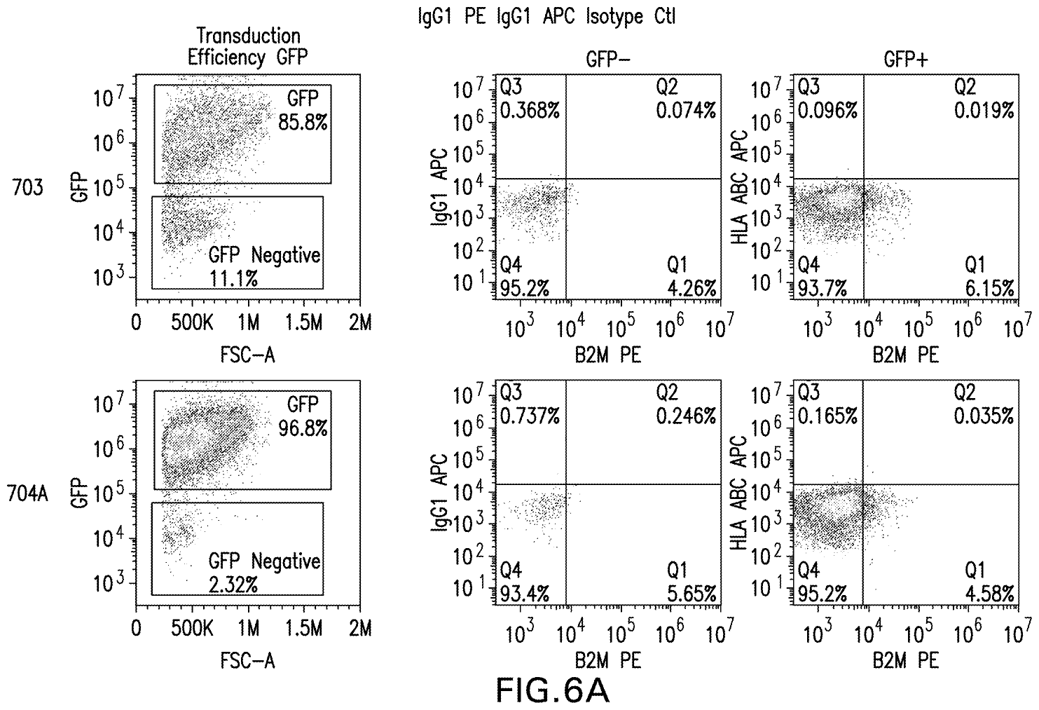

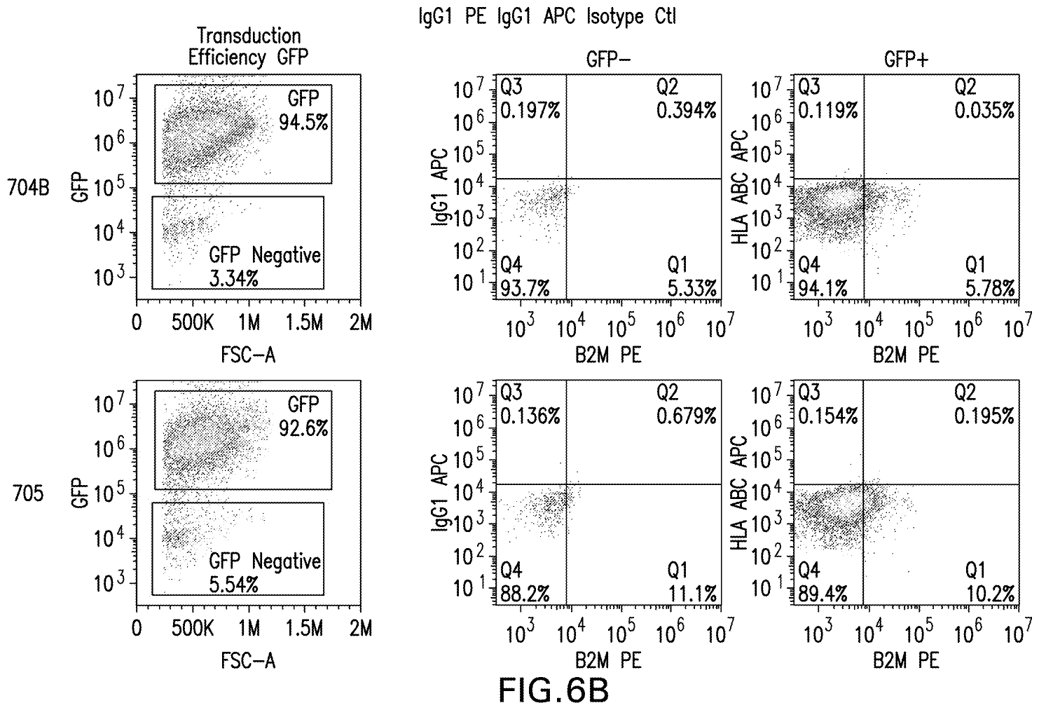

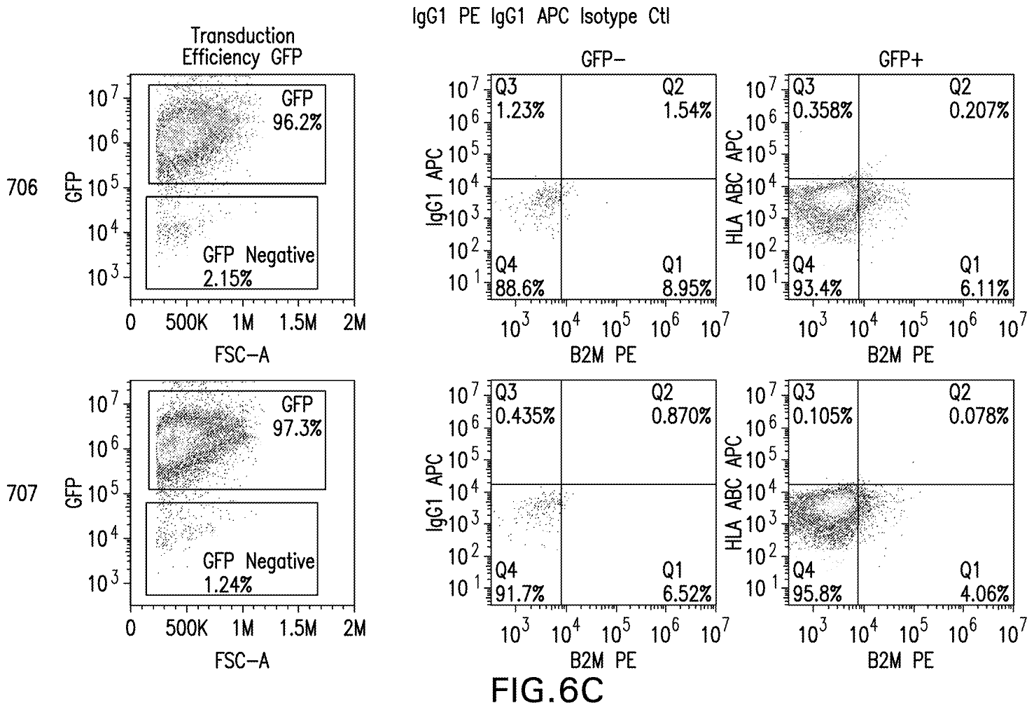

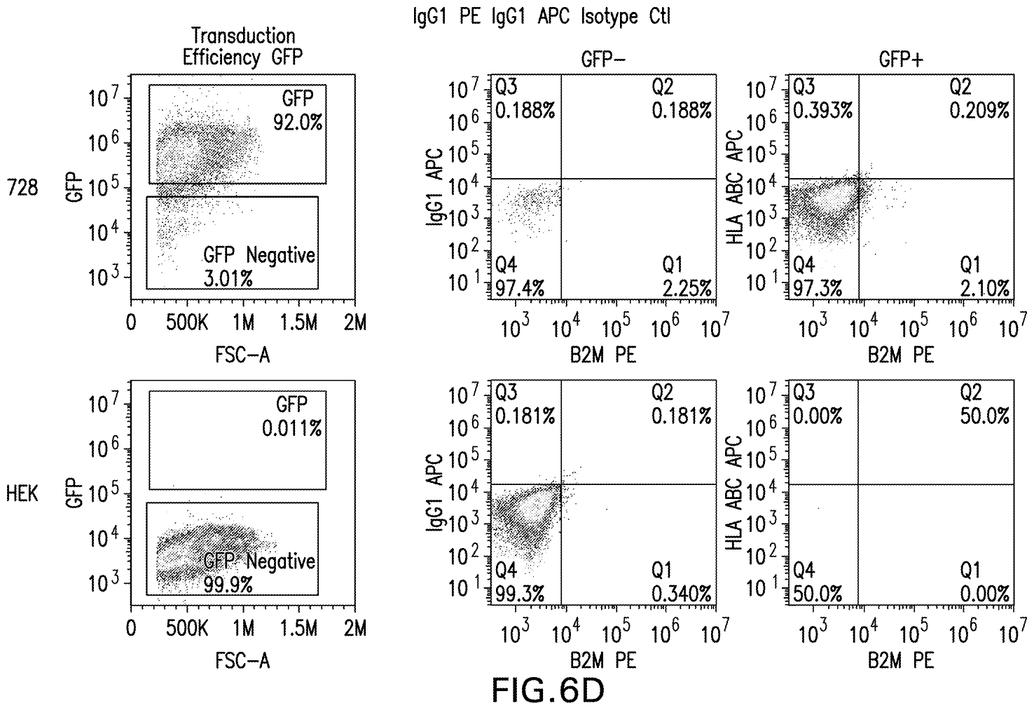

FIGS. 4A-B show mean fluorescent intensity for HEK-293 cells transduced with various shRNA-encoding, GFP+ vectors and stained with anti-B2M or anti-HLA ABC stains. FIGS. 5A-5D show GFP-measured transduction efficiency for HEK-293 cells transduced with various shRNA-encoding vectors and stained with anti-B2M and anti-HLA stains. FIGS. 6A-6D show GFP-measured transduction efficiency for HEK-293 cells transduced with various shRNA-encoding vectors and stained with an IgG1 PE IgG1 APC isotype control. The transduction efficiency and B2M knockdown effects of the vectors with siRNAs comprising SEQ ID NOS: 1-5 (703-707) are further supported by the results shown in FIGS. 4A-B, FIGS. 5A-D, and FIGS. 6A-D.

### EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

What is claimed is:

1. A vector encoding:
a beta-2 microglobulin (B2M) modifying RNA; and
a chimeric antigen receptor (CAR).

2. The vector of claim 1 wherein the vector is a lentiviral vector.

3. The vector of claim 1 wherein the B2M-modifying RNA comprises small interfering RNA (siRNA).

4. The vector of claim 3 wherein the siRNA is encoded by a sequence comprising one selected from the group consisting of SEQ ID NO: 1, SEQ ID NO: 2, SEQ ID NO: 3, SEQ ID NO: 4, and SEQ ID NO: 5.

5. The vector of claim 1 wherein the B2M-modifying RNA comprises small hairpin RNA (shRNA).

6. The vector of claim 5 wherein the shRNA is encoded by a sequence comprising one selected from the group consisting of SEQ ID NO: 1, SEQ ID NO: 2, SEQ ID NO: 3, SEQ ID NO: 4, and SEQ ID NO: 5 and one or more hairpin loop sequences encoded by a sequence comprising one selected from the group consisting of SEQ ID NO: 6 and SEQ ID NO: 7.

7. The vector of claim 1 operable to knock-down B2M expression in transduced cells by at least 75% compared to wild type.

8. The vector of claim 1 operable to knock-down B2M expression in transduced cells by at least 80% compared to wild type.

9. The vector of claim 1 operable to knock-down B2M expression in transduced cells by at least 85% compared to wild type.

10. The vector of claim 1 operable to knock-down B2M expression in transduced cells by at least 90% compared to wild type.

11. The vector of claim 1 operable to knock-down B2M expression in transduced cells by at least 95% compared to wild type.

12. The vector of claim 1 wherein the CAR specifically binds a glial cell marker.

13. The vector of claim 1 wherein the CAR specifically binds antigen presenting cell (APC) marker.

14. The vector of claim 1 wherein the CAR specifically binds a T helper 1 cell (Th1) marker.

15. The vector of claim 1 wherein the CAR specifically binds a T helper 17 cell (Th17) marker.

16. The vector of claim 1 wherein the CAR specifically binds to a marker specific to cells selected from the group consisting of pancreatic islet cells, pancreatic beta cells, cardiomyocytes, monocytes, macrophages, myeloid cells, intestinal cells, liver cells, kidney cells, kidney podocytes, kidney tubule cells, epithelial cells, salivary gland cells, lung cells, fibroblasts, connective tissue cells, Langerhans cells, keratinocytes, melanocytes, skin cells, hair follicle cells, and hair bulb cells.

17. The vector of claim 1, further encoding a suicide gene.

18. The vector of claim 1, further encoding a PET reporter gene.

19. The vector of claim 1, further encoding a TK suicide/PET reporter gene.

20. An engineered cell transduced with a vector encoding a beta-2 microglobulin (B2M) modifying RNA and a chimeric antigen receptor (CAR).

21. The engineered cell of claim 20 wherein the B2M-modifying RNA comprises small interfering RNA (siRNA).

22. The engineered cell of claim 21 wherein the siRNA is encoded by a sequence comprising one selected from the group consisting of SEQ ID NO: 1, SEQ ID NO: 2, SEQ ID NO: 3, SEQ ID NO: 4, and SEQ ID NO: 5.

23. The engineered cell of claim 20 wherein the B2M-modifying RNA comprises small hairpin RNA (shRNA).

24. The engineered cell of claim 23 wherein the shRNA is encoded by a sequence comprising one selected from the group consisting of SEQ ID NO: 1, SEQ ID NO: 2, SEQ ID NO: 3, SEQ ID NO: 4, and SEQ ID NO: 5 and one or more hairpin loop sequences encoded by a sequence comprising one selected from the group consisting of SEQ ID NO: 6 and SEQ ID NO: 7.

25. The engineered cell of claim 20 having a B2M expression of 25% or less that of an equivalent wild type cell.

26. The engineered cell of claim 20 having B2M expression of 20% or less that of an equivalent wild type cell.

27. The engineered cell of claim 20 having B2M expression of 15% or less that of an equivalent wild type cell.

28. The engineered cell of claim 20 having B2M expression of 10% or less that of an equivalent wild type cell.

29. The engineered cell of claim 20 having B2M expression of 5% or less that of an equivalent wild type cell.

30. The engineered cell of claim 20 wherein the CAR specifically binds a CNS glial cell marker.

31. The engineered cell of claim 20 wherein the CAR specifically binds antigen presenting cell (APC) marker.

32. The engineered cell of claim 20 wherein the CAR specifically binds a T helper 1 cell (Th1) marker.

33. The engineered cell of claim 20 wherein the CAR specifically binds a T helper 17 cell (Th17) marker.

34. The engineered cell of claim 20 wherein the CAR specifically binds to a marker specific to cells selected from the group consisting of pancreatic islet cells, pancreatic beta cells, cardiomyocytes, monocytes, macrophages, myeloid cells, intestinal cells, liver cells, kidney cells, kidney podocytes, kidney tubule cells, epithelial cells, salivary gland cells, lung cells, fibroblasts, connective tissue cells, Langerhans cells, keratinocytes, melanocytes, skin cells, hair follicle cells, and hair bulb cells, oligodendrocytes, astrocytes, microglial cells.

35. The engineered cell of claim 20 wherein the cell is selected from the group consisting of a stem cell and a lymphocyte.

36. The engineered cell of claim 20 wherein the cell is a regulatory T cell (Treg).

37. The engineered cell of claim 20 wherein the cell is derived from a donor for allogeneic transplant in a subject.

38. A method of treating an autoimmune or inflammatory disease in a subject, the method comprising administering to said subject a therapeutically effective amount of regulatory T cells (Tregs), each expressing a beta-2 microglobulin (B2M) modifying RNA and a chimeric antigen receptor (CAR) that specifically binds a ligand on a surface of a cell in a manner that suppresses an immune response in a subject, thereby treating the autoimmune or inflammatory disease in the subject.

39. The method of claim 38 wherein the B2M-modifying RNA comprises small interfering RNA (siRNA).

40. The method of claim 39 wherein the siRNA is encoded by a sequence comprising one selected from the group consisting of SEQ ID NO: 1, SEQ ID NO: 2, SEQ ID NO: 3, SEQ ID NO: 4, and SEQ ID NO: 5.

41. The method of claim 38 wherein the B2M-modifying RNA comprises small hairpin RNA (shRNA).

42. The method of claim 41 wherein the shRNA is encoded by a sequence comprising one selected from the group consisting of SEQ ID NO: 1, SEQ ID NO: 2, SEQ ID NO: 3, SEQ ID NO: 4, and SEQ ID NO: 5 and one or more hairpin loop sequences encoded by a sequence comprising one selected from the group consisting of SEQ ID NO: 6 and SEQ ID NO: 7.

43. The method of claim 38 wherein the Tregs have a B2M expression of 25% or less that of an wild type Treg.

44. The method of claim 38 wherein the subject is a human.

45. The method of claim 38 wherein the Tregs are not autologous.

46. The method of claim 38, wherein the cell is selected from the group consisting of a glial cell, an antigen presenting cell (APC), a T helper 1 cell (Th1), and a T helper 17 cell (Th17).

47. A vector encoding a beta-2 microglobulin (B2M) modifying RNA, the vector's sequence comprising one selected from the group consisting of: SEQ ID NO: 1, SEQ ID NO: 2, SEQ ID NO: 3, SEQ ID NO: 4, and SEQ ID NO: 5.

48. The vector of claim 47 wherein the vector is a lentiviral vector.

49. The vector of claim 47 wherein the B2M-modifying RNA comprises small interfering RNA (siRNA).

50. The vector of claim 47 wherein the B2M-modifying RNA comprises small hairpin RNA (shRNA).

51. The vector of claim 50 wherein the shRNA comprises one or more hairpin loop sequences encoded by a sequence comprising one selected from the group consisting of SEQ ID NO: 6 and SEQ ID NO: 7.

52. The vector of claim 47 operable to knock-down B2M expression in transduced cells by at least 75% compared to wild type.

53. The vector of claim 47 operable to knock-down B2M expression in transduced cells by at least 80% compared to wild type.

54. The vector of claim 47 operable to knock-down B2M expression in transduced cells by at least 85% compared to wild type.

55. The vector of claim 47 operable to knock-down B2M expression in transduced cells by at least 90% compared to wild type.

56. The vector of claim 47 operable to knock-down B2M expression in transduced cells by at least 95% compared to wild type.

57. The vector of claim 47, further encoding a suicide gene.

58. The vector of claim 47, further encoding a PET reporter gene.

59. The vector of claim 47, further encoding a TK suicide/PET reporter gene.

60. An engineered cell transduced with a vector encoding a beta-2 microglobulin (B2M) modifying RNA, the vector's sequence comprising one selected from the group consisting of: SEQ ID NO: 1, SEQ ID NO: 2, SEQ ID NO: 3, SEQ ID NO: 4, and SEQ ID NO: 5.

61. The engineered cell of claim 60 wherein the B2M-modifying RNA comprises small interfering RNA (siRNA).

62. The engineered cell of claim 60 wherein the B2M-modifying RNA comprises small hairpin RNA (shRNA).

63. The engineered cell of claim 62 wherein the shRNA comprises one or more hairpin loop sequences encoded by a sequence comprising one selected from the group consisting of SEQ ID NO: 6 and SEQ ID NO: 7.

64. The engineered cell of claim 60 having a B2M expression of 25% or less that of an equivalent wild type cell.

65. The engineered cell of claim 60 having B2M expression of 20% or less that of an equivalent wild type cell.

66. The engineered cell of claim 60 having B2M expression of 15% or less that of an equivalent wild type cell.

67. The engineered cell of claim 60 having B2M expression of 10% or less that of an equivalent wild type cell.

68. The engineered cell of claim 60 having B2M expression of 5% or less that of an equivalent wild type cell.

69. The engineered cell of claim 60 wherein the cell is selected from the group consisting of a stem cell and a lymphocyte.

70. The engineered cell of claim 60 wherein the cell is a regulatory T cell (Treg).

71. The engineered cell of claim 60 wherein the cell is derived from a donor for allogeneic transplant in a subject.

72. A method of treating an autoimmune or inflammatory disease in a subject, the method comprising administering to said subject a therapeutically effective amount of regulatory T cells (Tregs), each expressing a beta-2 microglobulin (B2M) modifying RNA in a manner that suppresses an immune response in a subject, thereby treating the autoimmune or inflammatory disease in the subject.

73. The method of claim 72 wherein the B2M-modifying RNA comprises small interfering RNA (siRNA).

74. The method of claim 73 wherein the siRNA is encoded by a sequence comprising one selected from the group consisting of SEQ ID NO: 1, SEQ ID NO: 2, SEQ ID NO: 3, SEQ ID NO: 4, and SEQ ID NO: 5.

75. The method of claim 72 wherein the B2M-modifying RNA comprises small hairpin RNA (shRNA).

76. The method of claim 75 wherein the shRNA is encoded by a sequence comprising one selected from the group consisting of SEQ ID NO: 1, SEQ ID NO: 2, SEQ ID NO: 3, SEQ ID NO: 4, and SEQ ID NO: 5 and one or more hairpin loop sequences encoded by a sequence comprising one selected from the group consisting of SEQ ID NO: 6 and SEQ ID NO: 7.

77. The method of claim 72 wherein the Tregs have a B2M expression of 25% or less that of a wild type Treg.

78. The method of claim 72 wherein the subject is a human.

79. The method of claim 72 wherein the Tregs are not autologous.


SEQUENCE LISTING (11 sequences)

SEQ ID NO: 1 - DNA, 21 bp, Artificial Sequence
  source: /note=“Description of Artificial Sequence Synthetic oligonucleotide”
gcagagaatggaaagtcaaat

SEQ ID NO: 2 - DNA, 21 bp, Artificial Sequence
  source: /note=“Description of Artificial Sequence Synthetic oligonucleotide”
attgaagttgacttactgaag

SEQ ID NO: 3 - DNA, 21 bp, Artificial Sequence
  source: /note=“Description of Artificial Sequence Synthetic oligonucleotide”
ggactggtctttctatctctt

SEQ ID NO: 4 - DNA, 21 bp, Artificial Sequence
  source: /note=“Description of Artificial Sequence Synthetic oligonucleotide”
gccgtgtgaaccatgtgactt

SEQ ID NO: 5 - DNA, 21 bp, Artificial Sequence
  source: /note=“Description of Artificial Sequence Synthetic oligonucleotide”
gtcaaatttcctgaattgcta

SEQ ID NO: 6 - DNA, 7 bp, Artificial Sequence
  source: /note=“Description of Artificial Sequence Synthetic oligonucleotide”
tcaagag

SEQ ID NO: 7 - DNA, 6 bp, Artificial Sequence
  source: /note=“Description of Artificial Sequence Synthetic oligonucleotide”
ctcgag

SEQ ID NO: 8 - DNA, 480 bp, Homo sapiens
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatac

SEQ ID NO: 9 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 10 - DNA, 1675 bp, Homo sapiens
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctga

SEQ ID NO: 11 - protein, 20 aa, Homo sapiens
MSRSVALAVLALLSLSGLEA